(19) United States
(12) Patent Application Publication
(10) Pub. No.: US 2015/0150790 A1
(43) Pub. Date: Jun. 4, 2015

(54) TRANSDERMAL ENHANCER

(71) Applicant: Jao Hung Biotechnology Co., Ltd., Taipei (TW)

(72) Inventor: Tsung-Min Hsu, Taipei (TW)

(21) Appl. No.: 14/552,568

(22) Filed: Nov. 25, 2014

Related U.S. Application Data
(60) Provisional application No. 61/911,557, filed on Dec. 4, 2013.

(51) Int. Cl. A61K 9/00; A61K 31/714; A61K 47/18; A61K 47/36; A61K 47/12; A61K 47/10; A61K 47/02; A61K 47/32; A61K 31/593; A61K 47/22

(52) U.S. Cl. CPC A61K 9/0014; A61K 31/593; A61K 31/714; A61K 47/183; A61K 47/22; A61K 47/12; A61K 47/10; A61K 47/02; A61K 47/186; A61K 47/32; A61K 47/36

(57) ABSTRACT

The present invention is related to a composition for enhancing topical and transdermal absorption comprising an alkali material having a pH in the range of about 9.0 to 14.0 and a water-soluble substance in an amount of more than 0.5 wt % based on a total weight of the composition. The present invention is also related to a pharmaceutical composition comprising the composition, at least one therapeutically effective amount of a drug or an effective cosmetic ingredient, and a pharmaceutically acceptable carrier. The present invention is also related to a method for enhancing and delivering a drug through a body surface to minimize or eliminate the possibility of skin damage, irritation or sensitization while the above-described composition or the pharmaceutical composition is crossing patient's skin or mucosal tissue.

### CROSS REFERENCE

This application claims priority to U.S. Provisional Patent Application Ser. No. 61/911,557, filed on Dec. 4, 2013, the content of which is hereby incorporated by reference in its entirety.

### BACKGROUND OF THE INVENTION

1. Field of the Invention

The present invention relates to topical and transdermal administration of pharmacologically active agents, and more particularly relates to a composition that enhances the permeability of skin or mucosal tissue to topically apply pharmacologically active agents.

2. Description of the Prior Arts

The deliveries of drugs through oral treatment or injection are inconvenient. For example, regarding oral treatment, some medicine may not endure the strong acid in stomach, or may even result in serious gastrointestinal side effects. Moreover, when some medicines are injected topically, irritation will occur. Thus, transdermal administration, a means of delivery of drugs through the skin, provides many advantages such as comfort, convenience and non-invasiveness.

However, skin is a structurally complex and thick membrane. While drugs or molecules are moving into deep skin, firstly, they must penetrate the stratum corneum, the epidermis, the dermis, and the capillary walls into the blood stream or lymph channels. To be absorbed, drugs or molecules must overcome different degrees of resistance for penetration into each type of cell layer. For instance, the stratum corneum is a thin layer of dense highly keratinized cells approximately 10-15 microns thick over most of the body. It is believed that the high degree of keratinization within these cells as well as their dense packing creates in most cases a substantially impermeable barrier for absorption.

Even though the prior arts have disclosed the water-soluble substance with the pH over 8.5 that helps the water-soluble substance get into the skin, they neither teach that the alkali material with the pH over 8.5 would cause irritation and corrosion of the stratum corneum layer of the skin, nor teach how to prevent and alleviate the irritation and corrosion caused by the alkali material with the pH over 9.

### SUMMARY OF THE INVENTION

To enhance the penetration of chemical materials, natural extract or organic polypeptides via the skin or the mucosa tissue of oral cavity, anus, and reproductive organs to body for treatment, health care or cosmetic purposes, the objective of the present invention is to provide a composition for enhancing topical and transdermal absorption.

To achieve the above objective, the composition comprises an alkali material having a pH in the range of about 9.0 to 14.0 and a water-soluble substance in an amount of more than 0.5 wt % based on a total weight of the composition.

The term “alkali material” as used herein refers to an agent that releases free hydroxide ions in an aqueous environment. Accordingly, any alkali material that has a pH value over 9.0 can open a channel between a protein portion (e.g. keratin) and a lipid bilayer at a stratum corneum layer of skin to permit water-soluble material with molecular weight less than 2,000 from penetrating into the body.

In one embodiment, example of the alkali material is selected from the group consisting of an organic base and an inorganic base, wherein the organic base includes a nitrogenous base. Preferably, the organic base includes, but is not limited to, trimethylamine. triethylamine, methylamine, ethylamine, dimethylamine, diethylamine, alkylamine, alkyldiamine, alkyltriamine, aromatic amine, tetramethylammonium hydroxide, tetraethylammonium hydroxide, tetrapropyl-ammonium hydroxide, tetramethylammonium bisulfide, tetraethylammonium bisulfide, pyridine and derivatives of pyridine, imidazole and derivatives of imidazole, benzimidazole and derivatives of benzimidazole, histidine and derivatives of histidine, phosphazene base, pydroxides of some organic cations, and combinations of two or more thereof. Wherein the aromatic amine includes, but is not limited to, aniline, toluidine, anisidine, and N-methylaniline. The inorganic base includes, but is not limited to, ammonium hydroxide, sodium hydroxide, potassium hydroxide lithium hydroxide, calcium hydroxide, magnesium hydroxide, cesium hydroxide, lithium carbonate, potassium carbonate, potassium phosphate, sodium carbonate, sodium phosphate, cesium carbonate and combinations thereof.

The term “water-soluble substance” as used herein refers to an agent that forms hydrogen bonding in an aqueous environment.

In another embodiment, example of the water-soluble substance includes, but is not limited to, compound posing amine functional group: primary amine, secondary amine, tertiary amine, and quaternary ammonium cations; compound posing carboxylic acid functional group: fatty acid, amino acid, keto acid, aromatic carboxylic acid, dicarboxylic acid, tricarboxylic acid, and alpha hydroxyl acid; compound posing sulfhydryl functional group: aliphatic and aromatic sulfhydryl compound; compound posing hydroxyl functional group: aliphatic and aromatic alcohol, glycols, triols, and polyals; compound posing phosphoric acid functional group: potassium phosphoric acid, sodium phosphoric acid, and benzylphosphoric acid. Preferably, the water-soluble substance is gelatin, agar, alginic acid, sodium alginate, dextrin, xanthan gum, gum arabic powder, hydroxyethylcellulose, hydroxypropylcellulose, hydroxypropylmethylcellulose, polyvinylpyrrolidone, partially saponified polyvinyl alcohol, methylcellulose, pullulan, partially pregelatinized starch, or saccharides. These substances may be used individually or in combination of two or more.

However, alkali material with the pH over 9.0 may hurt the skin, inasmuch as alkali material would release free hydroxide ions to result in irritation and corrosion due to the pH over 9. Therefore, the water-soluble substance of concentration more than 0.5 wt % of a total weight of the composition forms hydrogen bonding in the aqueous environment to decrease the activity of water and the degree of dissociation of the alkali material, and to reduce the activity of free hydroxide ion and avoid irritation and corrosion.

On the other hand, by means of composition having the water-soluble substance of more than 0.5 wt % and alkali material with the pH over 9.0, wherein the alkali material can constantly release free hydroxide ions to open a channel between the protein portion (e.g. keratin) and lipid bilayer at the stratum corneum layer of skin, and then the channel can constantly open to permit water-soluble material with molecular weight less than 2,000 through the body.

The advantage of the composition in accordance with the present invention not only clearly provides a mechanism of the alkali material affecting the region of the skin and the water-soluble substance penetration, but also provides a means to reduce harm caused by the alkali material. In brief, the alkali material with the pH over 9.0 of the composition can release free hydroxide ions in an aqueous environment, and the water-soluble substance of more than 0.5 wt % of the composition can form hydrogen bonding in an aqueous environment to enhance the penetration of substances such as drugs or molecules and to avoid irritation and corrosion of the skin. As for a water-insoluble substance, it could be dissolved in water with the addition of a solution such as alcohol to penetrate into the skin.

In a related aspect of the present invention, a pharmaceutical composition of matter is provided for delivering a drug through a body surface using the composition comprising alkali material with the pH over 9.0 and water-soluble substance with more than 0.5 wt % of the composition as a permeation enhancer. Generally, the formulation comprises (a) at least one therapeutically effective amount of a drug or an effective cosmetic ingredient, (b) alkali material with the pH over 9.0 and water-soluble substance in an amount effective to enhance the flux of the drug through the body surface without causing damage thereto, and (c) a pharmaceutically acceptable carrier suitable for topical or transdermal drug administration. The composition may be in any form suitable for application to the body surface, and may comprise, for example, a cream, lotion, solution, gel, ointment, paste or the like, and/or may be prepared so as to contain liposome, micelles, and/or microspheres. The composition may be directly applied to the body surface or may involve use of a drug delivery device. In one case, it is preferred although not essential that water be present in order for the water-soluble substance to form hydrogen bonding and thus decrease the activity of water and the degree of dissociation of the alkali material, to reduce the activity of free hydroxide ion and avoid irritation and corrosion. In another case, a formulation or drug reservoir may be aqueous, i.e., contain water, or may be nonaqueous and used in combination with an occlusive overlayer so that moisture evaporating from the body surface is maintained within the formulation or transdermal system during administration.

In another aspect of the present invention, a method is provided for minimizing or eliminating the possibility of skin damage, irritation or sensitization while the above-described composition is crossing patient's skin or mucosal tissue.

### BRIEF DESCRIPTION OF THE DRAWINGS

FIG. 1 shows effects of cream on treated skin indicated by Corneometer CM 825 within 7 days; wherein A is a cream from example 1 of the present invention, B is a commercially available cream of OLAY®, C is a commercially available cream of Clinique®, and D is the commercially available vaseline.

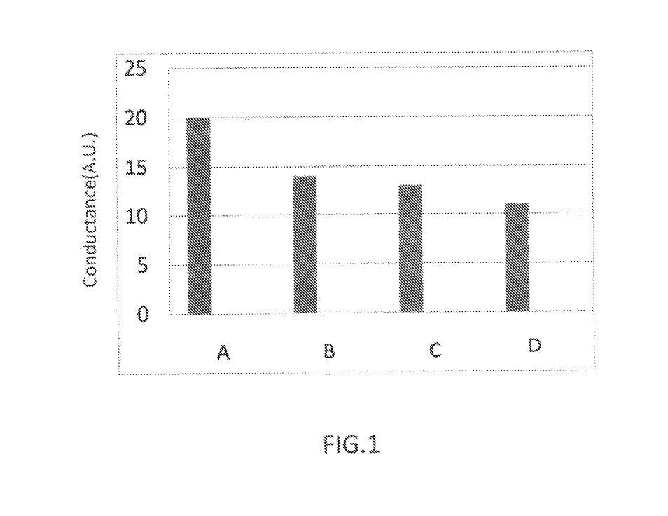

FIG. 2 shows effects of cream on treated skin indicated by transepidermal water loss (TEWL) within 7 days; wherein A is the cream from example 1 of the present invention, B is the commercially available cream of OLAY®, C is the commercially available cream of Clinique®, and D is the commercially available vaseline.

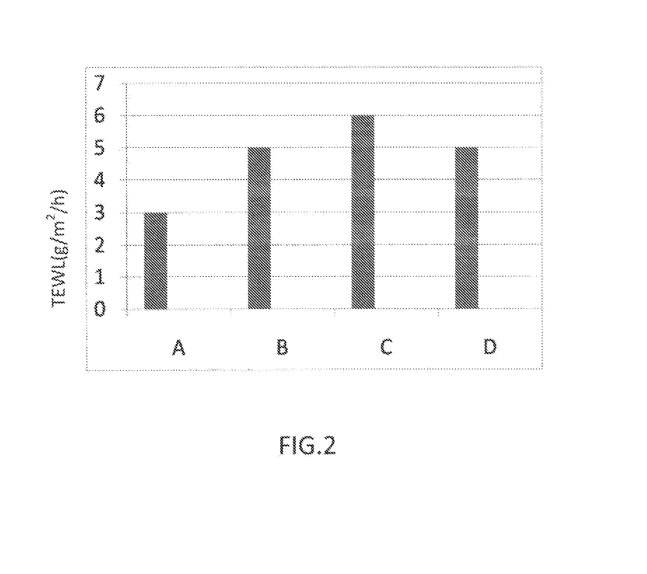

FIG. 3 is a measurement of D-Squame Scores on treated skin indicated within 7 days; and, wherein A is the cream from example 1 of the present invention, B is the commercially available cream of OLAY®, C is the commercially available cream of Clinique®, and D is the commercially available vaseline.

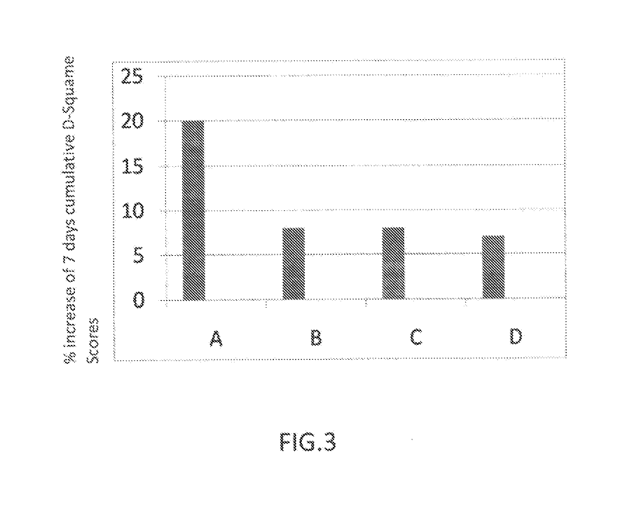

FIG. 4 shows the cumulative score of skin smoothness of 16 patients within 14 days, wherein A is the cream from example 1 of the present invention, B is the commercially available cream of OLAY®, C is the commercially available cream of Clinique®, D is the commercially available vaseline.

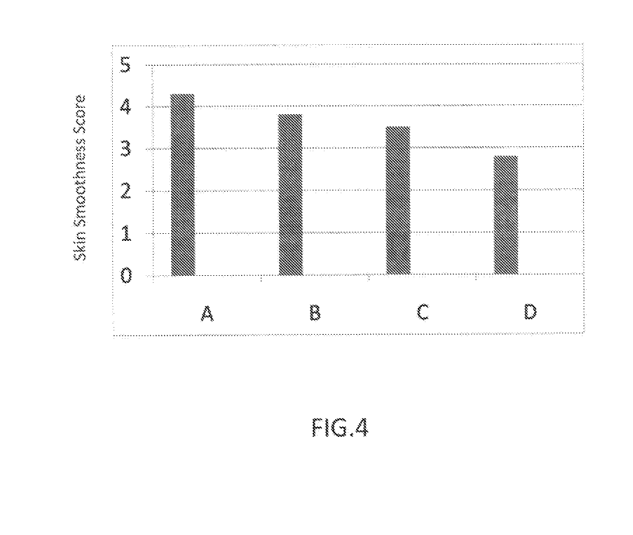

### DETAILED DESCRIPTION OF THE PREFERRED EMBODIMENTS

Other objectives, advantages and novel features of the invention will become more apparent from the following detailed description.

### Example 1

Cream, as also well known in the art, is viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. An in vitro skin permeation study was conducted using an oil-in-water cream which has pH value at 12.0 as shown in the following Table 1.

TABLE 1
Weight percent of each component (based on total solution weight)

| | |
|---|---|
| Distilled water | 72.8% |
| Ornithine HCl | 2.5% |
| Arginine | 2.7% |
| PCA-Na (sodium pyrrolidone carboxylate) | 2.3% |
| Lactic acid | 3.0% |
| Glycerin | 4.0% |
| NaOH (1:2 diluted with water) | 6.3% |
| Cetyl alcohol | 3.4% |
| Cetrimonium chloride | 2.0% |
| Brij 720 | 0.8% |
| Brij 30 | 0.2% |

### Example 2

Generally, the underlying surface of the transdermal device, i.e., the skin contact area, has an area in the range of about 5 cm² to 200 cm², preferably 5 cm² to 100 cm², and more preferably 20 cm²′ to60 cm². The area will vary with the amount of drug to be delivered and the flux of the drug through the body surface. Larger patches will be necessary to accommodate larger quantities of drug, while smaller patches can be used for smaller quantities of drug and/or drugs that exhibit a relatively high permeation rate.

An in-vitro skin permeation study was conducted using two vitamin D3 transdermal systems. The formulations used to prepare these systems are listed as shown in the following Table 2, which includes weight percent of each ingredient in the formulation.

TABLE 2
Weight percent of each
component based on total weight of each patch

| Vitamin D3 patch A | | Vitamin D3 patch B | |
|---|---|---|---|
| Vitamin D3 | 1.97% | Vitamin D3 | 1.97% |
| Diethylene | 14.80% | Diethylene | 9.87% |
| glycolmonoethylether | | glycolmonoethylether | |
| Polyvinyl pyrrolidone | 11.85% | Polyvinyl pyrrolidone | 6.90% |
| Polyisobutylene adhesive | 68.82% | Polyisobutylene adhesive | 56.26% |
| (Duro-tak ® 87-6908) | | (Duro-tak ® 87-6908) | |
| NaOH (1:2 diluted with | 1.58% | NaOH (1:2 diluted with | 3.00% |
| water) | | water) | |
| Sodium lauroylsarcosine | 0.99% | Lysine | 8.00% |
| | | Arginine | 8.00% |
| | | cetrimonium chloride | 3.00% |
| | | Brij 30 | 3.00% |

All ingredients were mixed together with Duro-tak®, Polyisobutylene adhesive, and laminated 50 mil.wet on a silicon coated releasing liner and cut into round discs. This 50 mil. thick patch was dried at 60° C. oven to remove n-heptane from the adhesive. The dried patch was then laminated with a polyethylene backing to form a vitamin D3 in matrix A patch having pH value at 11.0 and a vitamin D3 in matrix B patch having pH value at 13.5.

The in-vitro permeation of vitamin D3 through human cadaver skin from these discs was performed using Franz-type diffusion cells with a diffusion area of 1 cm². The volume of receiver solution was 8 ml. Human cadaver skin was cut to desired size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution. Twelve diffusion cells were used for each formulation. The measured skin permeations at the skin/patch interface were listed in Table 3.

TABLE 3
Skin permeation of vitamin D3 patch A and patch B

| | |
|---|---|
| vitamin D3 patch A | 1.9 (μg/cm²/24 hrs) |
| vitamin D3 patch B | 5.2 (μg/cm²/24 hrs) |

The cumulative amount of vitamin D3 of the patch A permeated across human cadaver skin at 24 hours is 1.9 μg/cm²/24 hrs; the cumulative amount of vitamin D3 of the patch A permeated across human cadaver skin at 24 hours is 5.2 μg/cm²/24 hrs.

### Example 3

Gels are semisolid, as will be appreciated by those working in the field of pharmaceutical formulation suspension-type systems. Single-phase gels contain organic macromolecules distributed substantially uniformly throughout the carrier liquid, which is typically aqueous, but also, preferably, contains an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

An in vitro skin permeation study was conducted using two vitamin B12 transdermal gels. The formulations used to prepare these gels are listed in Table 4, which includes weight of each component in the formulations.

TABLE 4
Weight of each component based on total weight of each
transdermal gel

| Vitamin B12 gel A* | | Vitamin B12 gel B | |
|---|---|---|---|
| Part I gel | | methylcobalamin | 0.5 mg |
| Methylcobalamin | 0.5 mg | hyaluronic acid | 0.5 g |
| hyaluronic acid | 0.5 g | lysine | 1.0 g |
| Lysine | 1.0 g | distilled water | 98 g |
| distilled water | 98 g | | |
| Part II gel | | | |
| NaOH (1:2 diluted with water) | 10.0 g | | |
| hyaluronic acid | 0.5 g | | |
| distilled water | 74.5 g | | |
| Lysine | 15.0 g | | |

*Mix Part I gel and Part II gel together before application on the skin.

The measured skin permeations at the skin/gel interface were listed in Table 5.

TABLE 5
Skin permeation of vitamin B12 gel A and gel B

| | |
|---|---|
| Vitamin B12 gel A | 0.09 (μg/cm²/24 hrs) |
| Vitamin B12 gel B | 0 (μg/cm²/24 hrs) |

The cumulative amount of vitamin B12 of the gel A permeated across human cadaver skin at 24 hours is 0.09 μg/cm²/24 hrs; the cumulative amount of vitamin B12 of the gel B permeated across human cadaver skin at 24 hours is 0 μg/cm²/24 hrs.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A composition comprising:
an alkali material having a pH in the range of about 9.0 to 14.0; and,
a water-soluble substance in an amount of more than 0.5 wt % based on a total weight of the composition.

2. The composition of claim 1, wherein the alkali material is selected from the group consisting of an organic base and an inorganic base.

3. The composition of claim 2, wherein the organic base has a nitrogenous base.

4. The composition of claim 3, wherein the organic base is selected from the group consisting of trimethylamine. triethylamine, methylamine, ethylamine, dimethylamine, diethylamine, alkylamine, alkyldiamine, alkyltriamine,aromatic amine, tetramethylammonium hydroxide, tetraethylammonium hydroxide, tetrapropyl- ammonium hydroxide, tetramethylammonium bisulfide, tetraethylammonium bisulfide, pyridine and derivatives of pyridine, imidazole and derivatives of imidazole, benzimidazole and derivatives of benzimidazole, histidine and derivatives of histidine, phosphazene base, pydroxides of organic cations, and combinations of two or more thereof.

5. The composition of claim 4, wherein the aromatic amine is selected from the group consisting of aniline, toluidine, anisidine, and N-methylaniline.

6. The composition of claim 2, wherein the inorganic base is selected from the group consisting of ammonium hydroxide, sodium hydroxide, potassium hydroxide lithium hydroxide, calcium hydroxide, magnesium hydroxide, cesium hydroxide, lithium carbonate, potassium carbonate, potassium phosphate, sodium carbonate, sodium phosphate, cesium carbonate and combinations thereof

7. The composition of claim 1, wherein the water-soluble substance is a compound posing amine functional group, carboxylic acid functional group, sulfhydryl functional group, hydroxyl functional group or phosphoric acid functional group.

8. The composition of claim 7, wherein the compound posing amine functional group is selected from the group consisting of primary amine, secondary amine, tertiary amine, and quaternary ammonium cation.

9. The composition of claim 7, wherein the compound posing carboxylic acid functional group is selected from the group consisting of fatty acid, amino acid, keto acid, aromatic carboxylic acid, dicarboxylic acid, tricarboxylic acid and alpha hydroxyl acid.

10. The composition of claim 7, wherein the compound posing sulfhydryl functional group is aliphatic or aromatic sulfhydryl compound.

11. The composition of claim 7, wherein the compound posing hydroxyl functional group is selected from the group consisting of aliphatic and aromatic alcohol, glycols, triols, and polyals.

12. The composition of claim 7, wherein the compound posing phosphoric acid functional group is selected from the group consisting of potassium phosphoric acid, sodium phosphoric acid, and benzylphosphoric acid.

13. The composition of claim 1, wherein the water-soluble substance is selected from the group consisting of gelatin, agar, alginic acid, sodium alginate, dextrin, xanthan gum, gum arabic powder, hydroxyethylcellulose, hydroxypropylcellulose, hydroxypropylmethylcellulose, polyvinylpyrrolidone, partially saponified polyvinyl alcohol, methylcellulose, pullulan, partially pregelatinized starch, and saccharides.

14. A pharmaceutical composition, comprising:
(a) at least one therapeutically effective amount of a drug or an effective cosmetic ingredient;
(b) the composition as claimed in claim 1; and,
(c) a pharmaceutically acceptable carrier suitable for topical or transdermal drug administration.

15. The pharmaceutical composition of claim 14, further comprising liposome, micelles or microspheres.

16. The pharmaceutical composition of claim 15, wherein formulation of the pharmaceutical composition is cream, lotion, solution, gel, ointment or paste.

17. A method for delivering a drug through a body surface, comprising administering the drug to a localized region of a human patient's body surface by the pharmaceutical composition as claimed in claim 14 to enhance the flux of the drug through the localized region of the body surface without causing damage thereto.

18. The method of claim 17, wherein the body surface is skin or mucosal tissue.
